(19) United States
(12) Patent Application Publication
(10) Pub. No.: US 2019/0069764 A1
(43) Pub. Date: Mar. 7, 2019

(54) MEDICAL OVERTUBE

(71) Applicant: OLYMPUS CORPORATION, Tokyo (JP)

(72) Inventors: Takumi ISODA, Tokyo (JP); Tatsutoshi HASHIMOTO, Tokyo (JP)

(73) Assignee: OLYMPUS CORPORATION, Tokyo (JP)

(21) Appl. No.: 16/172,957

(22) Filed: Oct. 29, 2018

Related U.S. Application Data
(63) Continuation of application No. PCT/JP2016/064155, filed on May 12, 2016.

(51) Int. Cl. A61B 1/00; A61B 1/012; A61B 1/018

(52) U.S. Cl. CPC A61B 1/00135; A61B 1/00071; A61B 1/01; A61B 1/018; A61B 1/0125

(57) ABSTRACT

A medical overtube includes a long insertion unit having distal and proximal end portions; a bending unit connected to the distal end portion; an operation unit disposed at a proximal end side of the insertion unit; and a linear member having a distal end fixed to a distal end side of the bending unit and disposed movably along a longitudinal axis of the insetion and bending units. The bending unit is bent by the linear member being pulled toward the proximal end portion in response to an operation of the operation unit, the bending unit has a plurality of channels through which medical devices are passed, and the linear member in the insertion unit is disposed at a position shifted in a circumferential direction and inward in a radial direction with respect to a position of the linear member disposed in at least a part of the bending unit.

### CROSS-REFERENCE TO RELATED APPLICATIONS

This is a continuation of International Application PCT/JP2016/064155 which is hereby incorporated by reference herein in its entirety.

### TECHNICAL FIELD

The present invention relates to a medical overtube.

### BACKGROUND ART

There is known a medical overtube having, at a distal end, a bending unit that can be bent when operated and a plurality of channels through which an endoscope and treatment tools are passed (for example, refer to PTL 1).

In this medical overtube, the channels are placed so that the treatment tools are shown in the lower part of the viewing area of the endoscope. Distal ends of four wires are fixed to the distal end of the bending unit so that the four wires respectively correspond to the up direction, the down direction, the right direction, and the left direction in the endoscopic image, and bending maneuverability is obtained by applying a pulling force to the proximal end side of the wire that bends the bending unit up, down, right, or left.

### CITATION LIST

### Patent Literature

### {PTL 1}

Japanese Translation of PCT International Application, Publication No. 2008-536552

### SUMMARY OF INVENTION

An aspect of the present invention provides a medical overtube that includes a long insertion unit having a distal end portion and a proximal end portion; a bending unit connected to the distal end portion of the insertion unit, the bending unit being configured to bend; an operation unit disposed at a proximal end side of the insertion unit; and a linear member having a distal end fixed to a distal end side of the bending unit, the linear member being disposed movably along a longitudinal axis of the insetion unit and the bending unit. The bending unit is bent by the linear member being pulled toward the proximal end portion of the insertion unit in response to an operation of the operation unit. The bending unit has a plurality of channels through which medical devices that have passed through the insertion unit are passed. The linear member in the insertion unit is disposed at a position shifted in a circumferential direction and inward in a radial direction with respect to a position of the linear member disposed in at least a part of the bending unit.

### BRIEF DESCRIPTION OF DRAWINGS

FIG. 1 is a diagram illustrating the overall structure of a medical overtube according to a first embodiment of the present invention.

FIG. 2A is a transverse sectional view illustrating a bending unit of the medical overtube illustrated in FIG. 1.

FIG. 2B is a transverse sectional view illustrating an insertion unit of the medical overtube illustrated in FIG. 1.

FIG. 3 is a transverse sectional view illustrating a reference example of an insertion unit of a medical overtube when second guide tubes in the insertion unit are placed at the same positions as the wire fixing positions in the bending unit.

FIG. 4A is a transverse sectional view illustrating a bending unit of a medical overtube according to a second embodiment of the present invention.

FIG. 4B is a transverse sectional view illustrating an insertion unit of the medical overtube illustrated in FIG. 4A.

FIG. 5 is a transverse sectional view of a modification of the insertion unit of the medical overtube illustrated in FIG. 4B.

FIG. 6A is a transverse sectional view illustrating a modification of the bending unit of the medical overtube illustrated in FIG. 1.

FIG. 6B is a transverse sectional view illustrating an insertion unit of the medical overtube illustrated in FIG. 6A.

FIG. 7A is a transverse sectional view illustrating a modification of the bending unit of the medical overtube illustrated in FIGS. 4A and 4B.

FIG. 7B is a transverse sectional view illustrating an insertion unit of the medical overtube illustrated in FIG. 7A.

FIG. 8A is a transverse sectional view illustrating a modification of the bending unit of the medical overtube illustrated in FIG. 7A.

FIG. 8B is a transverse sectional view illustrating an insertion unit of the medical overtube illustrated in FIG. 8A.

### DESCRIPTION OF EMBODIMENTS

A medical overtube 1 according to a first embodiment of the present invention will now be described with reference to the drawings.

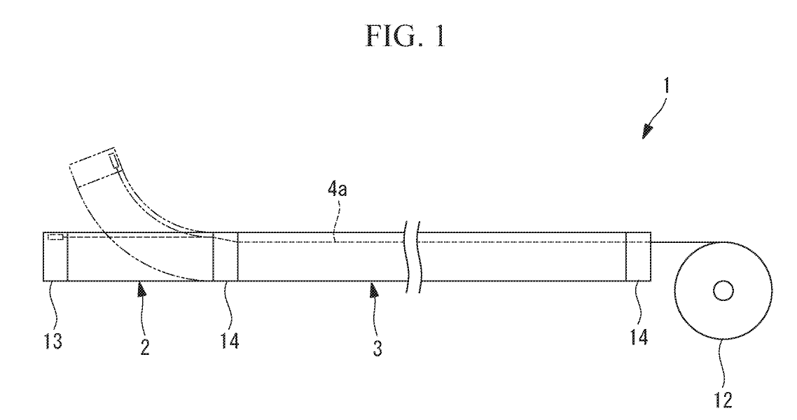

As illustrated in FIG. 1, the medical overtube 1 of this embodiment includes a bending unit 2 disposed at the distal end, a long insertion unit 3 connected to the proximal end side of the bending unit 2, and wires 4a, 4b, 4c, and 4d passing through the insertion unit 3 and the bending unit 2 in the longitudinal direction.

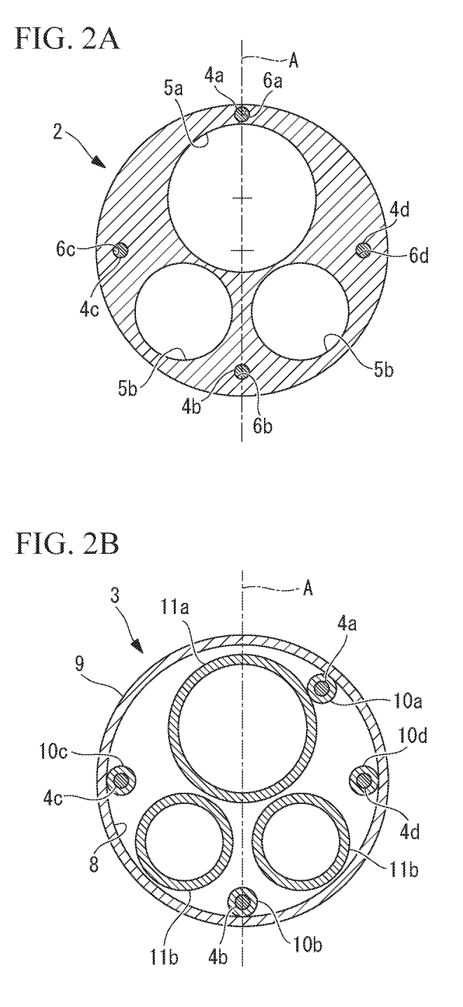

As illustrated in FIG. 2A, the bending unit 2 is formed of a multi-lumen tube, which is a tubular member composed of a flexible material and which has three channel lumens (channels) 5a and 5b, through which medical devices such as an endoscope and treatment tools are passed, and four wire lumens 6a, 6b, 6c, and 6d in which distal end portions of the wires 4a, 4b, 4c, and 4d are inserted.

The channel lumens 5a and 5b include one channel lumen (hereinafter may be referred to as a first lumen) 5a that has a relatively large diameter and forms an endoscope channel, and two channel lumens (hereinafter may be referred to as second lumens) 5b that have a diameter smaller than the first lumen 5a and through which treatment tools are passed. The first lumen 5a and the second lumens 5b are arranged in a positional relationship such that the two treatment tools protruding from distal end openings of the second lumens 5b are shown in a lower part in the viewing area of the endoscope exposed from the distal end opening of the first lumen 5a so that an overview image of the treatment tools from obliquely above can be acquired.

Thus, as illustrated in FIG. 2A, when the first lumen 5a is placed at the upper center part of the bending unit 2 and the two second lumens 5b are placed below the first lumen 5a, the operation of bending the bending unit 2 toward the first lumen 5a side along the plane A that includes the center line of the first lumen 5a and the center line of the bending unit 2 is called an upward bending operation, and, conversely, the operation of bending the bending unit 2 in the opposite direction is called a downward bending operation. The operation of bending the bending unit 2 in directions orthogonal to the plane A is called a horizontal bending operation.

The wire lumens 6a, 6b, 6c, and 6d are disposed at four positions: the top, the bottom, the left, and the right of the bending unit 2. The wire lumen 6a in which the first wire (linear member) 4a that bends the bending unit 2 upward is inserted is at the same circumferential position as the first lumen 5a, and is at a radially outward position with respect to the first lumen 5a. The wire lumens 6b, 6c, and 6d in which the wires 4b, 4c, and 4d are inserted are at circumferential positions different from that of any of the channel lumens 5a and 5b, and are thus placed radially inward with respect to the wire lumen 6a of the first wire 4a.

As illustrated in FIG. 2B, the insertion unit 3 includes a tubular outer casing tube 9 having an inner hole 8, four wire guide tubes (second guide tubes) 10a, 10b, 10c, and 10d, in the inner hole 8 of the outer casing tube 9, through which the wires 4a, 4b, 4c, and 4d are respectively passed, and three channel guide tubes (first guide tubes) 11a and 11b connected to the channel lumens 5a and 5b of the bending unit 2. Medical devices such as an endoscope and treatment tools are passed through the channel guide tubes 11a and 11b.

In the inner hole 8 of the outer casing tube 9, the channel guide tubes 11a and 11b are at substantially the same positions as the channel lumens 5a and 5b of the bending unit 2.

Of the wire guide tubes 10a, 10b, 10c, and 10d, the wire guide tube 10a through which the first wire 4a is passed is disposed at a position shifted in the circumferential direction and inward in the radial direction with respect to the position of the wire lumen 6a for the first wire 4a in the bending unit 2. The other wire guide tubes 10b, 10c, and 10d are at substantially the same positions as the wire lumens 6b, 6c, and 6d of the bending unit 2.

The wires 4a, 4b, 4c, and 4d having distal end portions inserted in the wire lumens 6a, 6b, 6c, and 6d of the bending unit 2 respectively pass through the wire guide tubes 10a, 10b, 10c, and 10d, are extracted from the proximal end side of the insertion unit 3, and are attached to the operation unit (proximal end portion) 12. Specifically, the operation unit 12 is formed of two pulleys for taking up the wires, and the wires 4a, 4b, 4c, and 4d are wound around the pulleys so that two wires are wound around one pulley.

When the wires 4a, 4b, 4c, and 4d are taken up by rotating the operation unit 12, tensions are applied to the proximal end portions of the wires 4a, 4b, 4c, and 4d. The applied tensions are transmitted to the bending unit 2 through the wires 4a, 4b, 4c, and 4d, and the bending unit 2 is bent in directions corresponding to the wires 4a, 4b, 4c, and 4d.

In FIG. 1, reference sign 13 denotes a fixing member (distal end portion) that fixes the distal ends of the wires 4a, 4b, 4c, and 4d to the distal end of the bending unit 2, and reference sign 14 denotes fixing members that are disposed at two ends of the insertion unit 3 and fix the two ends of the wire guide tubes 10a, 10b, 10c, and 10d and the channel guide tubes 11a and 11b.

The operation of the medical overtube 1 according to this embodiment configured as described above will now be described.

In order to use the medical overtube 1 of this embodiment, an endoscope is inserted from the proximal end side of the insertion unit 3 into one of the channel guide tubes 11a, 11b in the insertion unit 3 and into the channel lumen 5a connected to the channel guide tube 11a, and the distal end of the endoscope is exposed at the distal end of the bending unit 2. Treatment tools are respectively inserted from the proximal end side of the insertion unit 3 into the other two channel guide tubes 11b and into the channel lumens 5b connected to the channel guide tubes 11b, and the distal ends of the treatment tools are made to protrude from the distal end of the bending unit 2. The medical overtube 1 in this state is inserted into a body cavity of a patient, and the distal end is arranged to oppose the affected site.

In this manner, when the endoscope is actuated, the distal end portions of the treatment tools and the affected site in the body cavity are displayed in an overview image acquired through the endoscope from obliquely above. Here, the channel lumen 5a in which the endoscope is inserted is shown at the top and the channel lumens 5b in which the treatment tools are inserted are shown at the bottom.

If the operator wishes to move the viewing area range of the displayed image upward, the operator turns the operation unit 12 connected to the proximal end portion of the first wire 4a. As a result, a tension is applied to the first wire 4a, and the bending unit 2 bends upward. Similarly, if the operator wishes to move the viewing area range of the image in other directions, the operator applies tensions to the other wires 4b, 4c, and 4d fixed to the wire lumens 6b, 6c, and 6d placed in the corresponding directions so as to bend the bending unit 2 in the corresponding directions. Specifically, when the operator wishes to move the viewing area range downward, the operator applies a tension to the wire 4b; when the operator wishes to move the viewing area range to the left as viewed from the distal end side, the operator applies a tension to the wire 4c; and when the operator wishes to move the viewing area range to the right as viewed from the distal end side, the operator applies a tension to the wire 4d. As a result, the bending unit 2 bends in the corresponding direction.

As a result, the endoscope and the treatment tools can be moved simultaneously relative to the body tissue.

In this case, the medical overtube 1 of this embodiment offers an advantage in that, since the wire lumen 6a to which the first wire 4a is fixed is placed at a radially outward position with respect to the channel lumen 5a on the plane A that includes the center line of the channel lumen 5a for the endoscope and the center line of the bending unit 2, a maximum bending moment can be generated relative to the tension applied to the wire 4a, and the bending unit 2 can be bent upward with a small force.

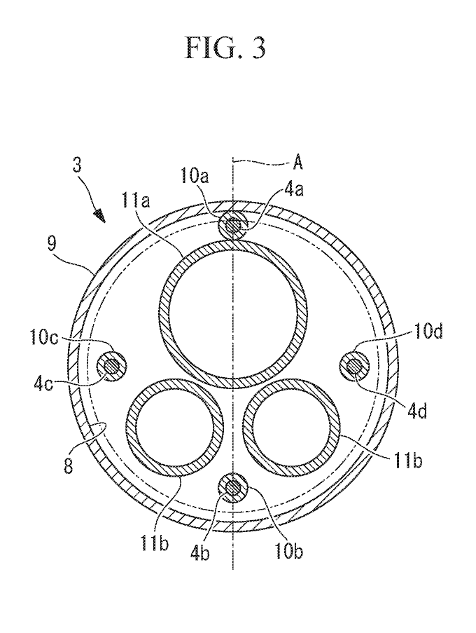

Moreover, there is also an advantage in that, since the wire guide tube 10a, through which the first wire 4a is passed, inside the inner hole 8 of the outer casing tube 9 of the insertion unit 3 is placed at a position shifted in the circumferential direction and the radial direction with respect to the wire lumen 6a to which the first wire 4a is fixed in the bending unit 2, the outer casing tube 9 can be made thin compared to the case in which the wire guide tube 10a is at the same position as illustrated in the reference diagram of FIG. 3. In FIG. 3, the two-dot chain line indicates the outer diameter of the medical overtube 1 of this embodiment.

That is, since the insertion unit 3 must be guided while preventing buckling against the tension applied to the wire 4a, a tube, such as a coil tube, having flexibility and high compressive strength is employed as the wire guide tube 10a inside the insertion unit 3. In the case where the coil tube is placed at the same position as illustrated in FIG. 3, since the coil tube has a larger outer diameter than the wire 4a, the outer diameter indicated by a solid line is needed for the outer casing tube 9 due to the necessity of placing the coil tube inside the inner hole 8 of the outer casing tube 9.

In contrast, as with the medical overtube 1 of this embodiment, when the wire guide tube 10a through which the first wire 4a passes is shifted in the circumferential direction and inward in the radial direction, the wire guide tube 10a can be placed in the gap formed between the channel guide tubes 11a and 11b. Thus, the inner hole 8 of the outer casing tube 9 can be arranged to be near the outer surfaces of the channel guide tubes 11a and 11b.

The outer diameter of the insertion unit 3 does not increase even when other wire guide tubes 10b, 10c, and 10d are placed at substantially the same positions as the wire lumens 6b, 6c, and 6d in the bending unit 2.

As mentioned above, the effects of the medical overtube 1 of this embodiment are that a large bending moment can be generated and the buckling of the insertion unit 3 can be prevented without increasing the outer diameter of the insertion unit 3, and that the diameter can be decreased while ensuring the bending maneuverability of the bending unit 2.

Next, a medical overtube according to a second embodiment of the present invention is described with reference to the drawings.

In the description of this embodiment, the structures that are common to those of the medical overtube 1 of the first embodiment described above are referred to by the same reference signs, and the descriptions therefor are omitted.

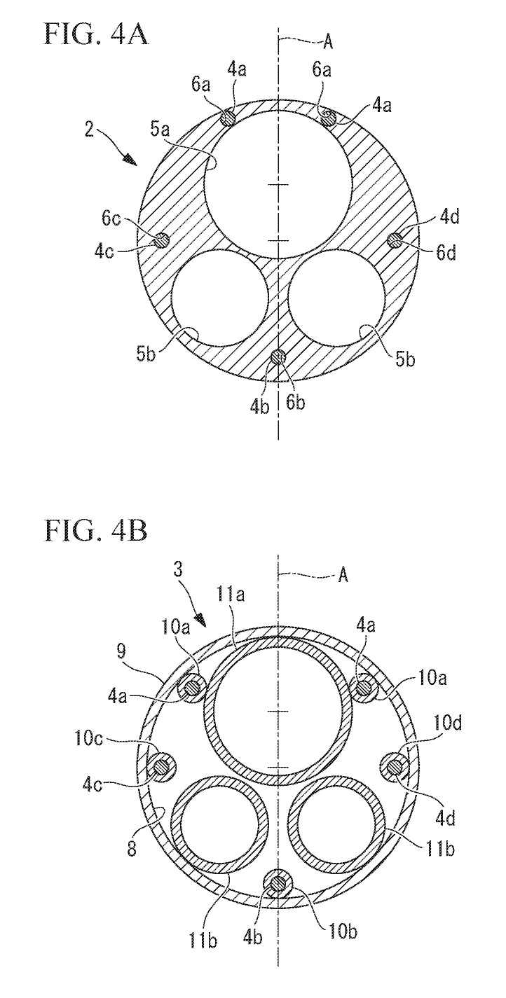

As illustrated in FIGS. 4A and 4B, the medical overtube of this embodiment differs from the medical overtube 1 of the first embodiment in that there are two first wires 4a for bending the bending unit 2 upward.

The two first wires 4a are respectively disposed on the two sides of the channel lumen 5a with the plane A, which includes the center line of the channel lumen 5a for the endoscope and the center line of the bending unit 2, therebetween.

In the medical overtube of this embodiment, when the bending unit 2 is to be bent upward in a direction along the plane A, an equal tension is applied to the two first wires 4a simultaneously. In this manner, since the first wires 4a are shifted in the circumferential direction from the plane A, the bending moments in the horizontal directions generated by the tensions acting on the first wires 4a are generated in opposite directions between the two first wires 4a and thus cancel each other out, and an upward bending moment can be effectively generated.

Since the two first wires 4a fixed to the bending unit 2 are at positions shifted from the plane A in the circumferential direction, the positions at which the first wires 4a are fixed can be shifted inward in the radial direction compared to the case in which one first wire 4a is placed on the plane A, and the outer diameter of the bending unit 2 can be further decreased.

In addition, there is an advantage in that the outer diameter of the insertion unit 3 can also be decreased by shifting the wire guide tubes 10a for the first wires 4a in the insertion unit 3 in the circumferential direction and the radial direction.

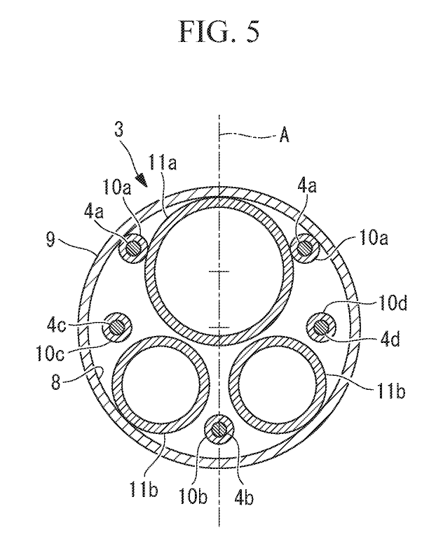

It should be noted that, as illustrated in FIG. 5, the wire guide tubes 10b, 10c, and 10d for the wires 4b, 4c, and 4d other than the first wire 4a can also be placed inward in the radial direction in the inner hole 8 of the outer casing tube 9 of the insertion unit 3.

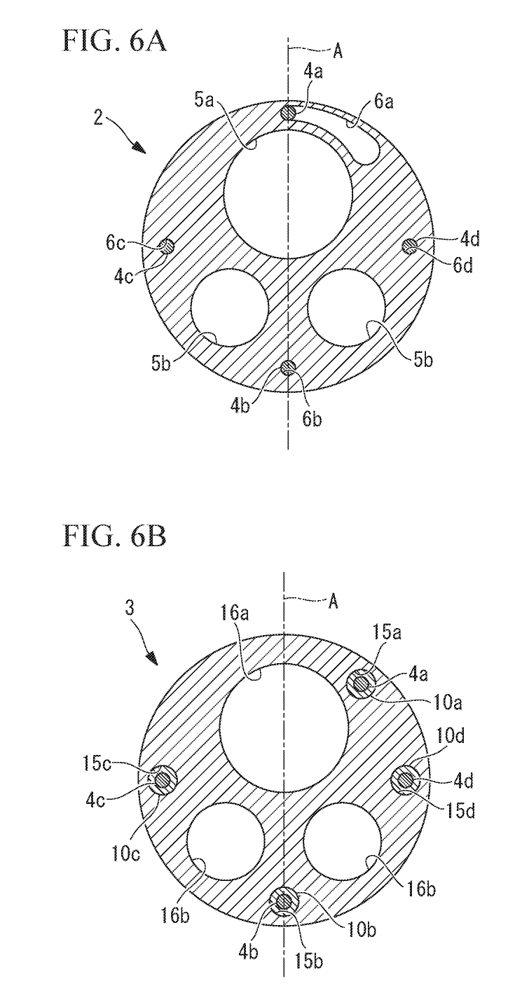

In this embodiment, as illustrated in FIG. 6A and 6B, the insertion unit 3 can also be formed of a multi-lumen tube. In this case, the wire lumen 6a of the bending unit 2 has an elongated transverse sectional shape so as to include the wire 4a fixing position and a wire lumen (lumen) 15a in the insertion unit 3. In this manner, the wire 4a can be smoothly bent inside the wire lumen 6a of the bending unit 2 from the wire 4a fixing position, which is the outermost radial position, in the bending unit 2 toward the wire lumen 15a of the insertion unit 3 placed at a position shifted in the circumferential direction and inward in the radial direction. In this case, wire lumens (lumens) 15b, 15c, and 15d in the insertion unit 3 are at the same positions as the wire lumens 6b, 6c, and 6d in the bending unit 2. Moreover, channel lumens (channels) 16a and 16b in the insertion unit 3 are at the same positions as the channel lumens 5a and 5b of the bending unit 2.

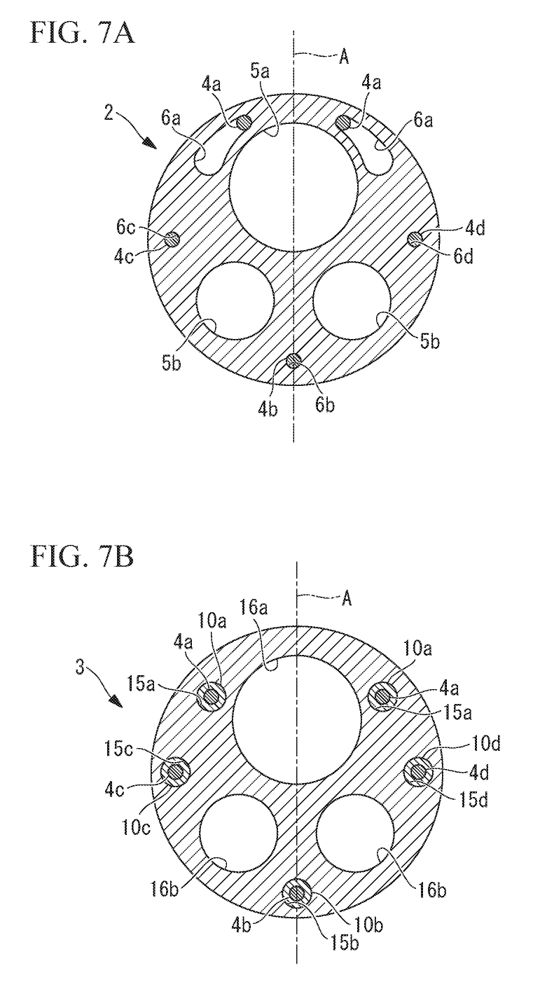

Moreover, even when there are two first wires 4a as illustrated in FIGS. 4A and 4B, the insertion unit 3 may be formed of a multi-lumen tube, as illustrated in FIGS. 7A and 7B. The wire lumens 6a for the two first wires 4a in the bending unit 2 preferably have an elongated transverse sectional shape as illustrated in FIGS. 6A and 6B.

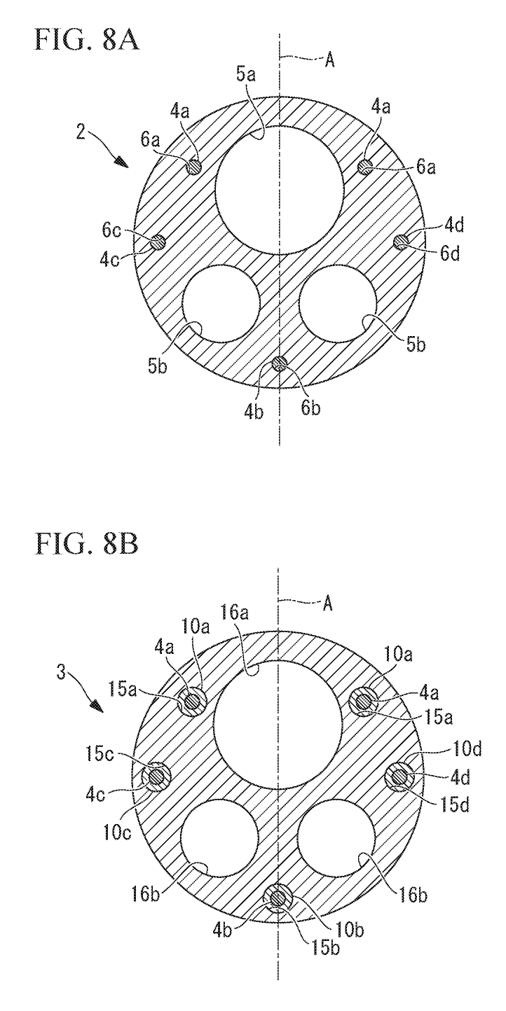

When there are two first wires 4a, as illustrated in FIGS. 8A and 8B, the first wire 4a fixing positions in the bending unit 2 may be the same as the positions of the wire lumens 15a in the insertion unit 3.

Alternatively, the wire lumens 15a of the insertion unit 3 may be formed to have an elongated shape so that the paths of the first wires 4a can be bent inside the insertion unit 3.

Although an example in which there are two first wires 4a is described, there may be three or more first wires 4a.

Moreover, the wires 4a, 4b, 4c, and 4d may each be a linear member formed by joining two or more linear members with different material properties. Furthermore, the part that is not bent may be partly formed of a rod.

The above-described embodiment also leads to the following invention.

An aspect of the present invention provides a medical overtube that includes a bending unit disposed at a distal end and that bends when operated; a long insertion unit connected to a proximal end side of the bending unit; a proximal end portion disposed at a proximal end side of the insertion unit; and a linear member disposed in the insertion unit so as to pass therethrough in a longitudinal direction, the linear member transmitting a tension applied to the proximal end portion so as to bend to the bending unit. The bending unit has a plurality of channels through which medical devices that have passed through the insertion unit can pass, and a radial position of the linear member in the insertion unit is radially inward with respect to a radial position of the linear member in the bending unit.

According to this aspect, when a tension is applied to the proximal end portion of the linear member at the proximal end side of the long insertion unit, the applied tension is transmitted through the linear member and the bending unit is bent. In this case, the linear member fixing position in the bending unit is preferably as radially outward as possible in order to generate a largest possible bending moment from the tension applied to the linear member. Meanwhile, the position of the linear member in the insertion unit is preferably adjusted to obtain a thickness necessary to obtain a compressive strength that resists buckling against the tension of the linear member.

According to this aspect, since the radial position of the linear member in the insertion unit is radially inward with respect to the linear member fixing position in the bending unit, a large moment can be generated in the bending unit without increasing the outer diameter of the insertion unit. In this manner, the diameter can be reduced while ensuring bending maneuverability of the bending unit.

In the aspect described above, the insertion unit may include an outer casing tube that has an inner hole, a plurality of first guide tubes that are disposed in the inner hole of the outer casing tube so as to pass therethrough in the longitudinal direction and that are connected to the channels of the bending unit, and a plurality of second guide tubes through which the linear member is passed, and at least one of the second guide tubes may be disposed at a position shifted in a circumferential direction and a radial direction with respect to a position of the linear member disposed in the bending unit.

In this manner, in the insertion unit, the linear member is guided by the second guide tube placed in the outer casing tube, and is fixed at a radially outward position with respect to the second guide tube in the bending unit. Moreover, since the second guide tube is shifted in the circumferential direction and the radial direction with respect to the linear member fixing position in the bending unit, the second guide tube can be placed in the gap formed between the first guide tubes inside the outer casing tube.

In this manner, the linear member fixing position in the bending unit can be as radially outward as possible so as to generate a large moment, and a compressive strength that can resist the tension applied to the linear member can be generated by the second guide tube. In this manner, the diameter can be reduced while ensuring bending maneuverability of the bending unit.

In the aspect described above, the bending unit and the insertion unit may each have a plurality of lumens through which the linear member is passed, at least one of the lumens may be disposed at a position shifted in a circumferential direction and a radial direction with respect to a position of the linear member disposed in the bending unit, and the lumen of the bending unit may have a transverse sectional shape that extends from a position where the linear member is fixed in the bending unit to the lumen in the insertion unit.

In this manner, in the insertion unit, the linear member is guided by the lumen placed at a radially inward position, and, in the bending unit, the linear member is fixed at a radially outward position with respect to the lumen in the insertion unit. Moreover, since the lumen in the insertion unit is shifted in the circumferential direction and the radial direction with respect to the linear member fixing position in the bending unit, the lumen for the linear member can be placed between the lumens formed in the insertion unit so as to connect to the channels in the bending unit.

Since the lumen in the bending unit has a transverse sectional shape that extends from the linear member fixing position to the lumen in the insertion unit, the linear member is inserted into the lumen in the insertion unit as the linear member bends in the circumferential direction and the radial direction inside the lumen in the bending unit. As a result, the linear member can smoothly pass through the lumens disposed at positions different in the circumferential direction and the radial direction.

In the aspect described above, the linear member that bends the bending unit in a direction along a plane that includes a center line of the bending unit and a center line of one of the channels may include two or more linear members disposed at positions flanking the plane.

In this manner, in bending the bending unit in a direction along the plane that includes the center line of one of the channels and the center line of the bending unit, tensions are simultaneously applied to the two or more linear members separated by the plane so as to cancel out the moments that work in directions orthogonal to the plane and to bend the bending unit in a direction along the plane. In this case, the linear member in the bending unit can be fixed at a radially inward position, and the diameter of the bending unit can be further reduced.

Moreover, in the aspect described above, the medical overtube may further include a distal end portion disposed at a distal end of the bending unit, the linear member may be a wire, and an end portion of the wire may be fixed to the distal end portion.

### REFERENCE SIGNS LIST

1 medical overtube
2 bending unit
3 insertion unit
4a first wire (linear member)
5a, 5b, 16a, 16b channel lumen (channel)
6a, 6b, 6c, 6d, 15a, 15b, 15c, 15d wire lumen (lumen)
8 inner hole
9 outer casing tube
10a, 10b, 10c, 10d wire guide tube (second guide tube)
11a, 11b channel guide tube (first guide tube)
12 operation unit (proximal end portion)
13 fixing member (distal end portion)
A plane

1. A medical overtube comprising:
a long insertion unit having a distal end portion and a proximal end portion;
a bending unit connected to the distal end portion of the insertion unit, the bending unit being configured to bend;
an operation unit disposed at a proximal end side of the insertion unit; and
a linear member having a distal end fixed to a distal end side of the bending unit, the linear member being disposed movably along a longitudinal axis of the insetion unit and the bending unit,
wherein the bending unit is bent by the linear member being pulled toward the proximal end portion of the insertion unit in response to an operation of the operation unit,
the bending unit has a plurality of channels through which medical devices that have passed through the insertion unit are passed, and
the linear member in the insertion unit is disposed at a position shifted in a circumferential direction and inward in a radial direction with respect to a position of the linear member disposed in at least a part of the bending unit.

2. The medical overtube according to claim 1, wherein the bending unit has the channel extending along the longitudinal axis,
the insertion unit includes an outer casing tube extending along the longitudinal axis, a first guide tube connected to the channel, and a second guide tube configured so that the linear member is inserted therein, and
the linear member passing through the second guide tube is disposed at a position shifted in the circumferential direction and inward in the radial direction with respect to a position of the linear member disposed in the bending unit.

3. The medical overtube according to claim 1, wherein the insertion unit has a lumen configured so that the linear member is inserted threrein, and
the linear member passing through the lumen is disposed at a position shifted in the circumferential direction and inward in the radial direction with respect to a position of the linear member disposed in the bending unit.

4. The medical overtube according to claim 2, wherein the outer casing tube has an inner hole extending along the longitudinal axis, and
the second guide tube is disposed along the longitudinal axis in the inner hole of the outer casing tube.

5. The medical overtube according to claim 1, wherein the linear member that bends the bending unit in a direction along a plane that includes a center line of the bending unit and a center line of the channel includes two or more linear members disposed at positions flanking the plane.

6. The medical overtube according to claim 1, further comprising:
a distal end portion disposed at a distal end of the bending unit,
wherein the linear member is a wire, and an end portion of the wire is fixed to the distal end portion.

7. The medical overtube according to claim 2, wherein the linear member that bends the bending unit in a direction along a plane that includes a center line of the bending unit and a center line of the channel includes two or more linear members disposed at positions flanking the plane.

8. The medical overtube according to claim 2, further comprising:
a distal end portion disposed at a distal end of the bending unit,
wherein the linear member is a wire, and an end portion of the wire is fixed to the distal end portion.

9. The medical overtube according to claim 3, wherein the linear member that bends the bending unit in a direction along a plane that includes a center line of the bending unit and a center line of the channel includes two or more linear members disposed at positions flanking the plane.

10. The medical overtube according to claim 3, further comprising:
a distal end portion disposed at a distal end of the bending unit,
wherein the linear member is a wire, and an end portion of the wire is fixed to the distal end portion.
